(19) United States
(12) Patent Application Publication
(10) Pub. No.: US 2011/0071510 A1
(43) Pub. Date: Mar. 24, 2011

(54) DEVICE FOR TREATMENT BY EMITTING FLASHES OF LIGHT

(75) Inventor: Georges Safraoui, Villejust (FR)

(73) Assignee: EUROFEEDBACK, Evry (FR)

(21) Appl. No.: 12/937,060

(22) PCT Filed: Apr. 10, 2009
(86) PCT No.: PCT/FR09/50650

§ 371 Date: Nov. 16, 2010

(30) Foreign Application Priority Data
Apr. 10, 2008 (FR) 0852427

(51) Int. Cl. A61B 18/18

(52) U.S. Cl. 606/9

(57) ABSTRACT

A device for treating the skin by emitting flashes of light, the device including a flash lamp, a dichroic filter for filtering the light emitted by the flash lamp, and an absorbent filter placed downstream from the dichroic filter. In the device, the dichroic filter is formed on the absorbent filter.

The present invention relates to devices for treatment by emitting flashes of light.

Such devices are used for example for epilation and they are described in particular in patent applications EP 1 602 340, EP 1 749 494, and European patent EP 1 547 538 in the name of the Applicant.

In general, the light emitted by the flash lamp has a broad spectrum and it needs to be filtered so that the spectrum of the light reaching the skin is adapted to the treatment to be performed.

In machines known by the Applicant, a red absorbent filter, e.g. of the Schott RG610 type, is placed between the flash lamp and a light guide of the handpiece taking the light to the skin.

The amount of light energy absorbed by the filter is relatively large because of its filtering in the blocked band and because of its efficiency being less than 100% in the passband. The rise in the temperature of the filter may lead to it being damaged.

Other manufacturers of devices for treatment by emitting flashes of light make use not of an absorbent filter but rather of a dichroic filter.

The advantage of a dichroic filter is reduced heating, thereby making it more robust.

The efficiency of such a filter is satisfactory only under normal incidence.

When the incident light is produced by a laser, the light reaches the filter perpendicularly and the filter therefore gives good results.

However, the Applicant has found that the efficiency of the filter is mediocre for filtering light emitted by a flash lamp, since that light reaches the filter at angles of incidence relative to the normal that are not negligible.

Consequently, the power emitted by flash lamp devices fitted with a dichroic filter cannot be as great as is desirable, because of the danger of burning the skin as a result of filtering that is not sufficiently selective.

EP 0 726 083 discloses a device including a flash lamp, an interchangeable interference filter, and an absorbent filter placed downstream from the interference filter.

In spite of the presence of the interference filter, the amount of energy received by the absorbent filter remains relatively large, thereby limiting the incident fluence. Unfortunately, in certain applications, it is desirable to have relatively high fluence.

There exists a need to further improve devices for treatment by flashes of light that include a flash lamp.

The invention seeks to satisfy this need, and does so by proposing a device that includes, downstream from the flash lamp, a dichroic filter for filtering the light emitted by the flash lamp, and downstream from the dichroic filter, an absorbent filter.

The dichroic filter serves to reduce significantly the amount of energy that is dissipated in the absorbent filter, but without that reducing the useful energy for performing the looked-for treatment.

The dichroic filter filters the light reaching the absorbent filter, thereby preventing it from receiving excessive light energy in its blocked band.

The dichroic filter is advantageously formed on the absorbent filter, e.g. by a technique of vacuum deposition on the absorbent filter, thereby enabling both the dichroic filter and the absorbent filter to be cooled effectively.

Furthermore, the use of a dichroic filter that is not sufficiently selective no longer constitutes a drawback because of the presence of the absorbent filter, and because of the possibility of cooling the absorbent filter, given that it is joined to the dichroic filter.

In particular, the dichroic filter may be in contact with a cooling liquid and the absorbent filter may be cooled through the dichroic filter in effective manner.

The invention makes it possible, where necessary, to increase the light energy emitted by the lamp without running the risk of excessively heating the absorbent filter, which might damage it, and without running the risk of burning the skin.

The light guide may be placed downstream from the absorbent filter, so as to convey the light leaving the absorbent filter towards the skin.

By way of example, and in particular for epilation, the dichroic filter may be arranged to eliminate wavelengths that are less than or equal to 550 nanometers (nm), better 600 nm, while passing wavelengths that are greater than or equal to 600 nm, better 610 nm.

In particular for epilation, the absorbent filter may be arranged to eliminate wavelengths less than or equal to 550 nm, better 600 nm, and to pass wavelengths greater than or equal to 600 nm, better 610 nm.

For other applications, the wavelengths may be different.

By way of example, the fluence of the light from the flash lamp upstream from the dichroic filter is greater than or equal to 40 joules per square centimeter (J/cm²) (e.g. for a 20 millisecond (ms) pulse), or even 100 J/cm², or even 150 J/cm².

By way of example, the duration of a flash of light may be less than or equal to 100 ms, better 50 ms, better still 1 ms.

The dichroic filter and the absorbent filter, in particular when the dichroic filter is formed on the absorbent filter, are advantageously cooled by circulating a liquid; e.g. the same liquid as is used for cooling the flash lamp.

The invention can be better understood on reading the following detailed description of a non-limiting embodiment of the invention, and on examining the accompanying drawing in which the sole figure is a diagrammatic view of an example of a device made in accordance with the invention.

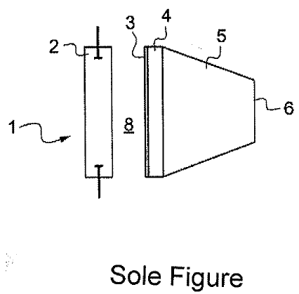

The device 1 for treatment by emitting flashes of light shown diagrammatically in FIG. 1 includes a flash lamp 2 that, in the example described, is a rectilinear flash tube, but that could have some other shape.

The light emitted by the flash lamp 2 is filtered by a dichroic filter 3 and by an absorbent filter 4 prior to reaching a light guide 5 that conveys the light towards an outlet window 6, which window directs the light towards the region for treatment.

The flash lamp 2 may be cooled by a liquid circulating in the space 8 between the lamp 2 and the dichroic filter 3.

Where appropriate, transparent glass may be placed between the flash lamp 2 and the dichroic filter 3.

By way of example, the cooling liquid is demineralized water.

In the example described, the dichroic filter 3 is formed directly on a face of the absorbent filter 4 by a vacuum deposition technique, the natures of the deposited layers and their thicknesses being selected to have the passband that is desired for the filter.

By way of example, the dichroic filter passes only wavelengths greater than or equal to 610 nm (λ_{c}=610 nm). The selectivity of the absorbent filter is for example such that it also passes only wavelengths greater than or equal to 610 nm (λ_{c}=610 nm).

The flash lamp may emit pulse light, e.g. at a rate of more than one flash per second, with the duration of each pulse lying in the range 1 ms to 300 ms, for example, and with a sequence of pulses comprising one to ten flashes on the same treated region, for example.

The fluence of the light reaching the dichroic filter may for example lie in range 40 J/cm² to 150 J/cm² (e.g. for a 20 ms pulse).

The fluence of the light reaching the skin may for example lie in the range 20 J/cm² to 75 J/cm².

Naturally, the invention is not limited to the example described above.

The dichroic filter and the absorption filter preferably have the same cutoff wavelength λ_{c}, to within ±50 nm, better to within ±20 nm, better still to within ±10 nm, preferably to within ±5 nm.

Depending on the power, the absorbent filter may be cooled by air instead of liquid, because of the presence of the dichroic filter. Nevertheless, cooling by liquid through the dichroic filter is preferable.

The expression “comprising a” should be understood as being synonymous with “comprising at least one”.

1. A device for treating the skin by emitting flashes of light, the device comprising:
a flash lamp;
a dichroic filter for filtering the light emitted by the flash lamp; and
an absorbent filter placed downstream from the dichroic filter;
the dichroic filter being formed on the absorbent filter.

2. A device according to claim 1, the dichroic and absorbent filters being cooled by circulating a liquid, the dichroic filter being in contact with the liquid and the absorbent filter being cooled through the dichroic filter.

3. A device according to claim 1, including a light guide downstream from the absorbent filter.

4. A device according to claim 1, the dichroic filter and the absorbent filter having the same cutoff wavelength to within ±50 nm.

5. A device according to claim 1, the dichroic filter being arranged to pass wavelengths greater than or equal to 600 nm.

6. A device according to claim 1, the absorbent filter being arranged to pass wavelengths greater than or equal to 600 nm.

7. A device according to claim 1, the fluence upstream from the dichroic filter being greater than or equal to 40 J/cm².

8. A device according to claim 1, the duration of a light pulse being less than or equal to 100 ms.

9. A device according to claim 2, the liquid cooling the dichroic and absorbent filters being the same as the liquid cooling the flash lamp.

10. A device according to claim 2, the cooling liquid being water.
